(19) United States
(12) Patent Application Publication
(10) Pub. No.: US 2026/0076632 A1
(43) Pub. Date: Mar. 19, 2026

(54) RADIOGRAPHIC IMAGING SYSTEM AND RADIOGRAPHIC IMAGING SUPPORT METHOD

(71) Applicant: KONICA MINOLTA, INC., Tokyo (JP)

(72) Inventors: Kazuya YOGI, Kawagoe-shi (JP); Satoshi HASEGAWA, Tokyo (JP)

(73) Assignee: KONICA MINOLTA, INC., Tokyo (JP)

(21) Appl. No.: 19/325,990

(22) Filed: Sep. 11, 2025

(30) Foreign Application Priority Data
Sep. 13, 2024 (JP) 2024-158860

(51) Int. Cl. A61B 6/46; A61B 6/00; A61B 6/04

(52) U.S. Cl. CPC A61B 6/461; A61B 6/04; A61B 6/4405

(57) ABSTRACT

Disclosed is a radiographic imaging system including: an optical camera that captures an optical image of a subject; a hardware processor that displays an imaged radiographic image; and a mobile terminal that is wirelessly communicable with the optical camera or the hardware processor. The mobile terminal includes a display that displays the optical image of the subject captured by the optical camera.

### CROSS-REFERENCE TO RELATED APPLICATIONS

The entire disclosure of Japanese Patent Application No. 2024-158860 filed on Sep. 13, 2024, is incorporated herein by reference in its entirety.

### BACKGROUND OF THE INVENTION

### Technical Field

The present disclosure relates to a radiographic imaging system and a radiographic imaging support method.

### Description of Related Art

In the past, X-ray imaging systems have been disclosed that determine whether a patient can be considered non-moving based on image information generated by optically imaging the patient, for the purpose of performing X-ray imaging with suitable positioning (see, for example, JP 2017-136299A).

### SUMMARY OF THE INVENTION

In the X-ray imaging system disclosed in JP 2017-136299A, when it is determined that the patient can be considered non-moving, the optical imaging is ended, and an operator moves to a control room and gives an instruction to perform X-ray imaging. As a result, in the above X-ray imaging system, even if the posture of the patient changes after the patient has been determined to be non-moving, the operator cannot confirm the change.

The present disclosure has been made in consideration of the above-described problem, and an object of the present disclosure is to perform radiographic imaging while checking the positioning of a patient.

To achieve at least one of the abovementioned objects, a radiographic imaging system reflecting one aspect of the present invention comprises: an optical camera that captures an optical image of a subject; a hardware processor that displays an imaged radiographic image; and a mobile terminal that is wirelessly communicable with the optical camera or the hardware processor, wherein the mobile terminal includes a display that displays the optical image of the subject captured by the optical camera.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a schematic configuration diagram illustrating a radiographic imaging system according to an embodiment of the present disclosure;

FIG. 2 is a block diagram illustrating a functional configuration of an FPD;

FIG. 3 is a block diagram illustrating a functional configuration of a medical cart;

FIG. 4 is a block diagram illustrating a functional configuration of a tablet;

FIG. 5 is a flowchart illustrating a flow of an examination process;

FIG. 6 is a flowchart illustrating a flow of the examination process;

FIG. 7 is a flowchart illustrating a flow of the examination process;

FIG. 8 is a diagram illustrating a display of an examination screen; and

FIG. 9 is a diagram illustrating a display of the examination screen.

### DETAILED DESCRIPTION

Hereinafter, one or more embodiments of the present invention will be described with reference to the drawings. However, the scope of the invention is not limited to the disclosed embodiments.

### <1. Configuration of Radiographic Imaging System>

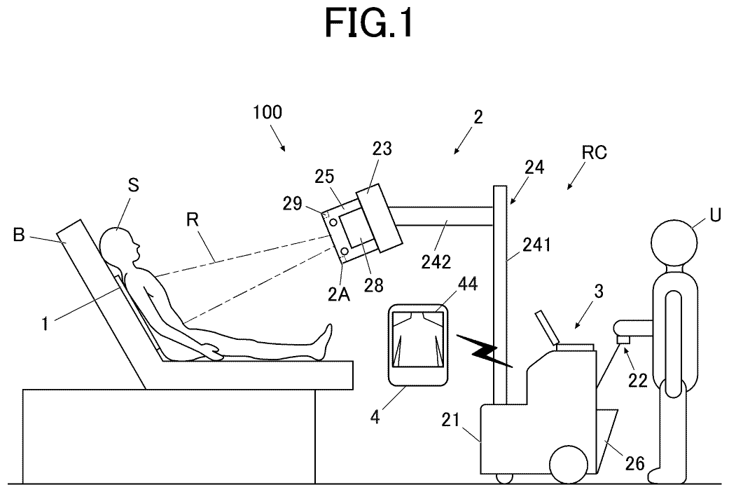

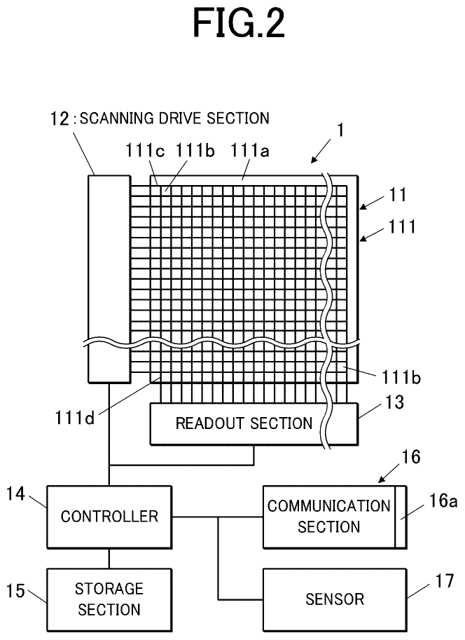

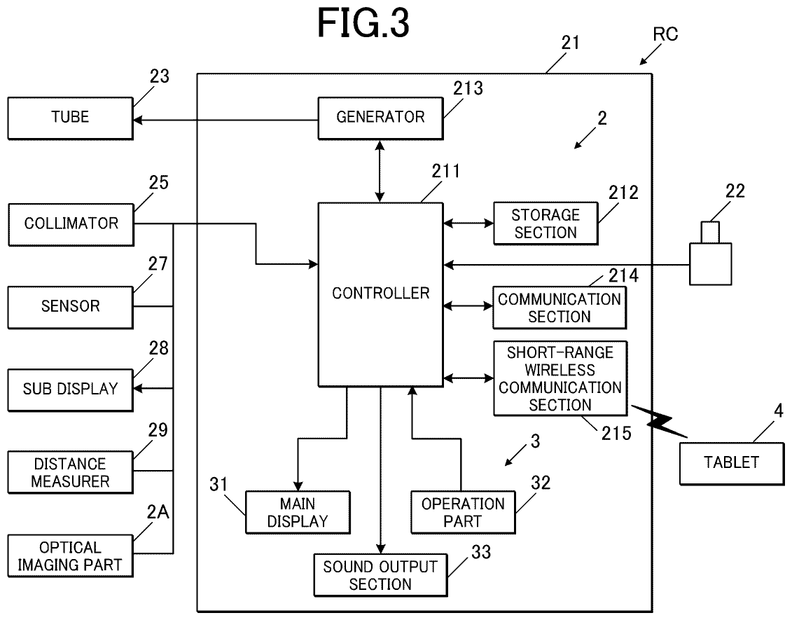

First, a schematic configuration of a radiographic imaging system according to the present embodiment will be described. FIG. 1 is a schematic configuration diagram illustrating a radiographic imaging system 100. FIG. 2 is a block diagram illustrating a configuration of a flat panel detector (FPD) 1. FIG. 3 is a block diagram illustrating a configuration of a medical cart RC.

As illustrated in FIG. 1, the radiographic imaging system (to be referred to as a system hereinafter) 100 according to the present embodiment is installed in a medical facility such as a hospital. A user U such as a radiographer of the medical facility moves, together with the system 100, to a medical examination destination such as a hospital ward or an operation room where a patient is located during rounds to perform radiographic imaging of the patient. Here, an example will be described in which the medical examination destination is a hospital ward, a bed B is installed in the hospital ward, and radiographic imaging is performed while a patient subject S is in a supine position on the bed B that is angled to raise the patient's upper body.

The system 100 includes the FPD 1, the medical cart RC, and a tablet 4. The medical cart RC includes a radiation generator (hardware processor) 2 and a console 3. The FPD 1 and the medical cart RC are communicable with each other, for example, via wireless communication. The medical cart RC can be communicably connected to a communication network (such as a local area network (LAN)) in the medical facility at a waiting place (storage place) before the rounds. The medical cart RC and the tablet 4 are connected to each other by short-range wireless communication. This short-range wireless communication is, for example, short-range wireless communication by Bluetooth® Low Energy (BLE). The tablet 4 is a mobile terminal used by the user U.

Note that the system 100 is not limited to the one that includes the medical cart RC and is movable by the medical cart RC but may be one installed in an imaging room of the medical facility. The system 100 may be capable of communicating with a hospital information system (HIS), a radiology information system (RIS), and/or the like. The system 100 may also be capable of communicating with a picture archiving and communication system (PACS) and/or a dynamic analysis apparatus. The communication network may be a wired network or a wireless network.

The FPD 1 generates radiographic image data according to radiation R emitted from the radiation generator 2. The FPD 1 is formed in a panel shape and can be carried. Therefore, the FPD 1 can be used not only by being mounted on an imaging table but also by being horizontally placed between the bed B and the subject S who is in a supine position on the bed B. Furthermore, as illustrated in FIG. 1, the FPD 1 can be used by being placed upright between the subject S in a sitting posture on the bed B, which is angled to raise the subject's upper body, or a wheelchair and the backrest of the bed B or wheelchair.

Note that the radiation-incident surface (the surface facing the subject S) of the FPD 1 mounted on an imaging table is parallel or perpendicular to the horizontal plane. In imaging without using an imaging table (e.g., on a bed or wheelchair), the radiation-incident surface may not necessarily be parallel or perpendicular to the horizontal plane (may be inclined). Further, when the FPD 1 is interposed between a soft instrument, such as the bed B, and the subject S, the FPD 1 may move along with the movement of the subject S.

The radiation generator 2 includes a generator main body 21, an irradiation instruction switch 22, a tube 23, a tube support portion 24, a collimator 25, and an FPD storage 26. The radiation generator 2 is movable with wheels provided on a housing of the generator main body 21.

The irradiation instruction switch 22 outputs an operation signal to the generator main body 21 in response to being operated (pressed) by the user U. Although FIG. 1 illustrates the irradiation instruction switch 22 connected to the generator main body 21 by a wire, the irradiation instruction switch 22 may be wirelessly connected to the generator main body 21.

In response to the irradiation instruction switch 22 being operated, the tube 23 generates radiation R (X-rays or the like) of a dose corresponding to preset imaging conditions in a mode corresponding to the imaging conditions and emits the radiation R from an irradiation port.

The tube support portion 24 is an arm that supports the tube 23. The tube support portion 24 includes a support portion 241 extending upward from the generator main body 21 to its upper end, and a support portion 242 extending forward from an upper portion of the support portion 241. An end portion of the support portion 242 supports the tube 23. The tube support portion 24 can move the tube 23 in the X-axis direction, the Y-axis direction orthogonal to the X-axis, and the Z-axis direction orthogonal to the X-axis and the Y-axis. The X-axis direction is the front-rear direction of the radiation generator 2, which is the left-right direction in FIG. 1. The Y-axis direction is the width direction of the radiation generator 2, which is the direction orthogonal to the plane of FIG. 1. The Z-axis direction is the vertical direction, which is the up-down direction in FIG. 1. The tube support portion 24 can change the direction of the irradiation port of the radiation R by rotating the tube 23 on rotation axes parallel to the X-axis, the Y-axis, and the Z-axis by a joint mechanism not illustrated.

The collimator 25 is attached to the irradiation port of the tube 23 and narrows the radiation R so that the irradiation field of the radiation R emitted from the irradiation port becomes a preset rectangular shape. The collimator 25 includes a lamp button (not illustrated). In response to the lamp button being operated by the user, the collimator 25 emits visible light to a range corresponding to the irradiation field of the radiation R.

The FPD storage 26 stores the FPD 1 not in use and is disposed on the side of the generator main body 21. The FPD storage 26 is capable of storing a plurality of FPDs 1. A connector (not illustrated) may be provided to the FPD storage unit 26, and when the FPD 1 is stored, the connector may be connected to a connector 16a (see FIG. 2) of the FPD 1.

The console 3 includes a personal computer (PC), a portable terminal, or a dedicated device. The console 3 is mounted on the radiation generator 2. The console 3 can set imaging conditions for at least one of the FPD 1 and the radiation generator 2 based on an imaging order acquired from an external device, such as the RIS, or an operation performed on an operation part 32 by the user U. The imaging conditions include a tube voltage, the product of a tube current and an irradiation time or a current time (mAs value), an imaging region, an imaging direction, and the like. The imaging order is information on the radiographic imaging that a clinician requests the user U to perform and includes the specified date and time of the radiographic imaging, subject information (e.g., patient ID) of the subject to be imaged, imaging region (e.g., imaging region ID), purpose ID, imaging content information, and the like. The console 3 can acquire radiographic image data generated by the FPD 1 and store the radiographic image data in the console 3 itself and/or transmit the radiographic image data to another external device (e.g., the PACS).

Radiographic imaging (imaging of a subject in a sitting posture) using the system 100 (medical cart RC) configured as described above is performed as follows. First, the user U places the system 100 near the subject S (beside the bed B). Then, the user U has the subject S take a sitting posture. When the subject S sits on an angle-adjustable instrument (e.g, the bed B that can be partially inclined), the user U adjusts the angle of the backrest of the bed B as appropriate. The user U roughly adjusts the position and orientation of the tube 23 so that the irradiation port of the tube 23 is directed toward the imaging region of the subject S. The user U takes out the FPD 1 from the FPD storage 26 and places the FPD 1 between the back of the subject S and the backrest. The user U finely adjusts the orientation and the irradiation field of the tube 23 so that the irradiation axis of the radiations R is orthogonal to the radiation-incident surface of the FPD 1. Then, the user U performs radiographic imaging. That is, the user U irradiates the imaging region of the subject S with the radiation R and causes the FPD 1 to generate radiographic image data of a still or dynamic image that shows the diagnosis target region.

Here, in performing the radiographic imaging, the user U needs to assist the subject S in positioning himself/herself so that the subject S does not move. As a result, since the user U is present adjacent to the bed B on which the subject S lies, the user U cannot see a main display 31 (see FIG. 3) of the console 3. Therefore, even when an optical image of the subject S imaged by an optical imaging part 2A described below is displayed on the main display 31, the user U cannot see the optical image. In the present embodiment, in order to solve such a problem, the optical image of the subject S imaged by the optical imaging part 2A is displayed on a display 44 of the tablet 4.

This enables the user U to check the positioning of the subject S while assisting the subject S in positioning himself/herself.

Note that the generator main body 21 and the console 3 are formed as a single unit (may be stored in one housing) but may be formed as separate units. The radiation generator 2 may be movable by means other than the wheels. For example, the radiation generator 2 may be reduced in weight so that the radiation generator 2 can be carried by a person or can be mounted on a commercially available cart or the like. The radiation generator 2 may have a smooth bottom surface so as to slide on a floor surface. Furthermore, in the system 100, one of the FPD 1 and the radiation generator 2 may be installed in a room or the like of the medical facility (while the other is freely movable).

Next, a functional configuration of the FPD 1 will be described with reference to FIG. 2. As illustrated in FIG. 2, the FPD 1 includes a radiation detector 11, a scanning drive section 12, a readout section 13, a controller 14, a storage section 15, a communication section 16, and a sensor 17. The components of the FPD 1 are connected to each other for communication.

The radiation detector 11 includes a scintillator (not illustrated) and a photoelectric conversion panel 111. The scintillator has a flat plate shape and is made of columnar crystals of CsI, for example. Upon receiving radiation, the scintillator emits electromagnetic waves (e.g., visible light) having a wavelength longer than that of the radiation, at an intensity corresponding to the dose (mAs) of the received radiation. The scintillator is also disposed so as to extend parallel to the radiation-incident surface of a housing of the radiation detector 11.

The photoelectric conversion panel 111 is disposed on the side of the scintillator opposite the side facing the radiation-incident surface and extends parallel to the scintillator. The photoelectric conversion panel 111 includes a circuit board 111a and a plurality of charge accumulation units 111b. On the surface of the circuit board facing the scintillator, the plurality of charge accumulation units 111b are two-dimensionally aligned (e.g., in a matrix) corresponding to the pixels of a radiographic image. Each of the charge accumulation units 111b includes a semiconductor element that generates an amount of charge corresponding to the intensity of the electromagnetic wave generated by the scintillator and a switch element that is disposed between the semiconductor element and a wiring connected to the readout section 13. The semiconductor element receives a bias voltage from a power supply circuit (not illustrated). Then, each of the charge accumulation units 111b accumulates and releases charges to be read out as a signal value corresponding to the received radiation by switching the switch element between an ON state and an OFF state.

The scanning drive section 12 switches the switching element to the ON state or the OFF state by applying an ON voltage or an OFF voltage, respectively, to a scanning line 111c of the radiation detector 11.

The readout section 13 reads out, as signal values, the amount of charge flowing in from the respective charge accumulation units 111b via respective signal lines 111d of the radiation detector 11. Note that the readout section 13 may perform binning when reading out the signal values.

The controller 14 includes a central processing unit (CPU) and a random-access memory (RAM), which are not illustrated. The CPU reads various processing programs stored in the storage section 15, loads the programs in the RAM, and executes various kinds of processing in cooperation with the loaded processing programs, thereby comprehensively controlling the operations of respective components of the FPD 1. The controller 14 also generates radiographic image data based on a plurality of signal values read out by the readout section 13.

The storage section 15 includes a semiconductor memory, a hard disk drive (HDD), or the like and stores the various programs to be executed by the controller 14, parameters necessary for executing the programs, and various data of files.

Note that the storage section 15 may be capable of storing image data of a radiographic image.

The communication section 16 includes a communication module for wireless communication, and the like. The communication section 16 transmits and receives various signals and data to and from an external device, such as the medical cart RC, wirelessly connected thereto.

The sensor 17 is a detector of information necessary for calculating the angle between the FPD 1 and the tube 23. The sensor 17 is a three-axis acceleration sensor. The three-axis acceleration sensor detects acceleration acting in each of the three axial (X-axis, Y-axis, and Z-axis) directions and outputs acceleration information on the detected acceleration in the three axis directions to the controller 14. In a stationary state, only the gravitational acceleration acts on the three-axis acceleration sensor. Therefore, in the stationary state, the three-axis acceleration sensor detects the components of the gravitational acceleration in the three axis directions.

Note that the sensor 17 may be a six-axis sensor or a nine-axis sensor. The six-axis sensor is a three-axis acceleration sensor with the addition of a function to detect angular velocity (gyro) in each of the three axis directions. The nine-axis sensor is a six-axis sensor with the addition of a function to detect azimuth (east, west, north, and south) in each of the three axis directions.

For example, in response to a predetermined condition being met, the controller 14 causes the sensor 17 to repeatedly detect the acceleration information on the gravitational acceleration in the three axis directions. Examples of the predetermined condition include that the power of the FPD 1 is turned on, that a predetermined control signal is received from another device (e.g., the medical cart RC), and that a predetermined operation is performed on an operation part (not illustrated) of the FPD 1. Each time the sensor 17 detects the acceleration information on the gravitational acceleration, the controller 14 transmits the detected acceleration information on the gravitational acceleration to the medical cart RC via the communication section 16.

As the operation of the controller 14, for example, the controller 14 causes the scan drive section 12 to accumulate and release charges in and from the radiation detector 11 in synchronization with the timing at which the radiation R is emitted from the radiation generator 2. The controller 14 also causes the readout section 13 to read out signal values based on the charges released by the radiation detector 11. In addition, the controller 14 generates radiographic image data of a still image or a moving image corresponding to the dose distribution of the emitted radiation R based on the signal values read out by the readout section 13. The moving image includes a dynamic image and a fluoroscopic image. When generating radiographic image data of a still image, the controller 14 generates the radiographic image data only once for each pressing of the irradiation instruction switch 22. When generating radiographic image data of a moving image, that is, when performing moving image imaging, the controller 14 repeatedly generates radiographic image data of a frame image constituting the moving image a plurality of times per predetermined time (e.g., 15 times per second) for each pressing of the irradiation instruction switch 22. The controller 14 transmits the generated radiographic image data to an external device (e.g., the medical cart RC) via the communication section 16.

Note that the FPD 1 is not limited to an indirect conversion type that converts radiation into an electrical signal via a scintillator, as described above, but may be a direct conversion type that directly converts radiation into an electrical signal by a semiconductor element.

Next, a functional configuration of the radiation generator 2 and console 3 of the medical cart RC will be described with reference to FIG. 3. As illustrated in FIG. 3, the radiation generator 2 includes a sensor 27, a sub display 28, a distance measurer 29, and the optical imaging part 2A in addition to the generator main body 21, the irradiation instruction switch 22, the tube 23, the tube support portion 24, the collimator 25, and the FPD storage section 26. The generator main body 21 further includes a controller 211, a storage section 212, a generator 213, a communication section 214, and a short-range wireless communication section 215. The components of the radiation generator 2, except for the tube 23, are communicably connected to each other.

The sensor 27 is provided in the tube 23 and is a three-axis acceleration sensor similar to the sensor 17. Note that the sensor 27 may be a six-axis sensor or a nine-axis sensor. In addition, the sensor 27 may be of a different type from the sensor 17. The controller 211 calculates the angle between (the radiation-incident surface of) the FPD 1 and (the surface perpendicular to the radiation-irradiation direction of) the tube 23 based on the acceleration information on the gravitational acceleration in the three axis directions received from the stationary FPD 1 via the communication section 214 and acceleration information on the gravitational acceleration in the three axis directions detected by the sensor 27 in the stationary tube 23.

The sub display 28 includes a display such as a liquid crystal display (LCD) or an electro-luminescence (EL) display and is disposed, for example, adjacent to the tube 23. The sub display 28 displays display information such as various images in accordance with display information input from the controller 211. The sub display 28 and the optical imaging part 2A are disposed on a housing of the collimator 25. Note that the sub display 28 and the optical imaging part 2A may be disposed on a housing of the tube 23 or on the tube support portion 24. In addition, the display content of the sub display 28 can be separated from the display content of the main display 31.

The distance measurer 29 measures a source image distance (SID), and outputs the measured SID to the controller 211. The SID is the distance between the focal point F of the radiation R and the imaging surface of the FPD 1 (the surface on which the charge accumulation units 111b of the radiation detector 11 are disposed). Note that the distance measurer 29 may be configured to measure a source skin distance (SSD). The SSD is the distance between the focal point F of the radiation R and the body surface of the subject S. The SSD is substantially equal to a difference between the SID and the body thickness of the subject S. The distance measurer 29 is provided to the collimator 25.

For example, the distance measurer 29 may include a light emission means that emits laser light, a detection means that detects reflected laser light, and a calculation means that calculates a distance from the light emission means to the reflection point based on a time from the emission of the laser light to the detection of the reflected laser light. For another example, the distance measurer 29 may include a calculation means that calculates the SID based on the optical image of the FPD 1, which is generated by the optical imaging part 2A that optically images the FPD 1 located at the irradiation direction, and size information of the FPD 1. The distance measurer 29 may be a combination of the above two examples. Since the laser light is reflected on the body surface of the subject S, the distance measured by the distance measurer 29 using the laser light is often the SSD. In this case, the sum of the measured SSD and the body thickness of the subject S is set as the SID. The body thickness may be a predetermined reference value, a numerical value input by the user U, or a value automatically calculated from information on the subject S.

The controller 211 calculates alignment information that includes: (inclination information (posture) of the radiation-incident surface of the FPD 1 with respect to the horizontal plane calculated from) the information on the three-axis acceleration of the FPD 1 provided by the sensor 17; (inclination information (posture) of the surface perpendicular to the radiation-irradiation direction of the tube 23 (collimator 25) with respect to the horizontal plane calculated from) the information on the three-axis acceleration of the tube 23 provided by the sensor 27; and the distance between the FPD 1 and the tube 23 measured by the distance measurer 29. The inclination information of the FPD 1 may be represented by a difference value from the inclination information of the tube 23 (collimator 25). The user can adjust the arrangement of the FPD1 and the tube 23 (collimator 25) by identifying the arrangement of the FPD1 and the tube 23 in the current radiographic imaging based on the alignment information in the past radiographic imaging. Note that the alignment information may be a single alignment of only the inclination information of the tube 23.

The optical imaging part 2A includes an optical system, such as a lens, and an imaging element, such as a charge coupled device (CCD) or a complementary metal oxide semiconductor (CMOS). Under the control of the controller 211, the optical imaging part 2A optically images the subject S as a subject with visible light to generate optical image data and outputs the optical image data to the controller 211 and the like. For example, the optical imaging part 2A optically images the subject S to generate optical image data such as a still image or a live image.

The controller 211 includes a CPU, a RAM, and the like. The CPU reads various programs stored in the storage section 212, loads the programs in the RAM, executes various processes in cooperation with the loaded programs, and controls the components of the radiation generator 2 and the console 3.

The storage section 212 includes a nonvolatile semiconductor memory, an HDD, or the like and stores various programs to be executed by the controller 211, and various types of data, such as parameters necessary for executing the programs, and files.

In response to receiving an imaging instruction signal from the controller 211, the generator 213 applies a voltage corresponding to the preset imaging conditions to the tube 23 and applies a current corresponding to the imaging conditions to the tube 23.

The communication section 214 includes a communication module and the like. The communication section 214 can transmit and receive various signals and various data to and from the FPD 1 connected wirelessly and an external device such as the RIS via a communication network.

Similarly to the communication section 214, the short-range wireless communication section 215 includes a communication module and the like. The short-range wireless communication section 215 performs short-range wireless communication with the tablet 4 via BLE.

The console 3 includes a controller, a storage section, a communication section, a main display 31, an operation part 32, and a sound output section 33. The controller 211, the storage section 212, and the communication section 214 of the radiation generator 2 serve as the controller, the storage section, and the communication section of the console 3, respectively. Note that the console 3 may include a dedicated controller, storage section, and communication section.

The main display 31 includes an LCD, an EL display, or the like. The main display 31 displays various kinds of information in accordance with display information input from the controller 211.

The operation part 32 includes, for example, a keyboard having various keys, a pointing device for inputting position information, a touch screen integrally formed on a display screen of the main display 31, and the like. The operation part 32 receives an operation input from the user U and outputs the operation information to the controller 211.

The sound output section 33 includes an amplifier, a speaker, and the like, and outputs sound in accordance with sound information input from the controller 211.

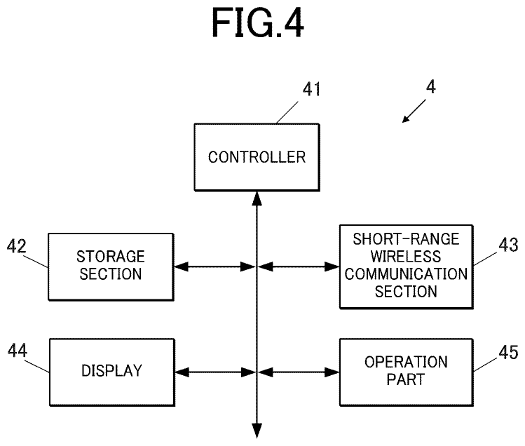

Next, a functional configuration of the tablet 4 will be described with reference to FIG. 4. As illustrated in FIG. 4, the tablet 4 includes a controller 41, a storage section 42, a short-range wireless communication section 43, a display 44, and an operation part 45.

The controller 41 includes a CPU, a RAM, and the like, and centrally controls the operation of each component of the tablet 4.

The storage section 42 includes a nonvolatile memory, a hard disk, or the like, and stores various programs to be executed by the CPU, parameters necessary for executing the programs, and the like.

The short-range wireless communication section 43 includes a communication module and the like. The short-range wireless communication section 43 performs short-range wireless communication with the medical cart RC (radiation generator 2) via BLE.

The display 44 includes, for example, an LCD, and the like. The display 44 displays an image (for example, an optical image of the subject S imaged by the optical imaging part 2A) corresponding to image data acquired from the controller 41.

The operation part 45 includes various function keys, a touch screen laminated on the surface of the display 44, and the like. The operation part 45 outputs a control signal corresponding to an operation performed by the user U to the controller 41.

### <2. Operation of Radiographic Imaging System>

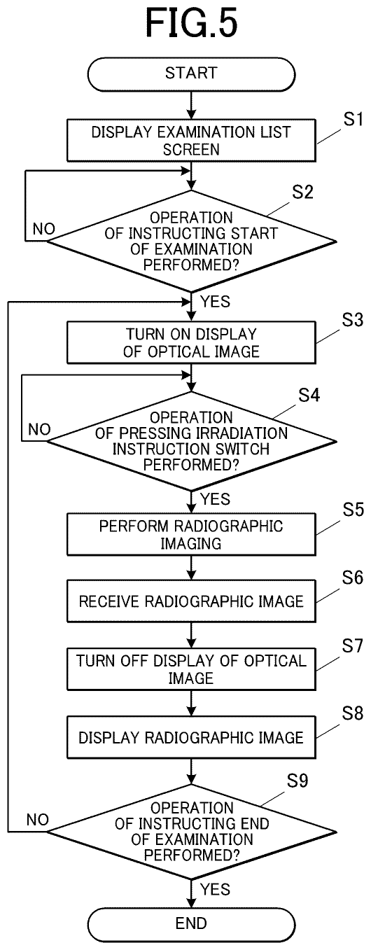

Next, an operation flow of an examination process executed by the system 100 will be described with reference to FIG. 5. Note that this examination process can also be applied to the case where the system 100 is installed in an imaging room of the medical facility as described above.

As illustrated in FIG. 5, when the examination process is started, first, the controller 211 displays an examination list screen (not shown) on the main display 31 (step S1). The examination list screen includes an examination list display field for displaying information on examination orders in a list, an examination information display field for displaying information on an examination order that has been selected via the operation part 32, an examination start button for instructing the start of an examination, and the like.

Next, the controller 211 determines whether an operation of instructing the start of the examination, that is, pressing the examination start button described above has been performed (step S2). If the controller 211 determines in step S2 that the operation of instructing the start of the examination has not been performed (step S2; NO), the controller 211 repeatedly performs the determination process of step S2.

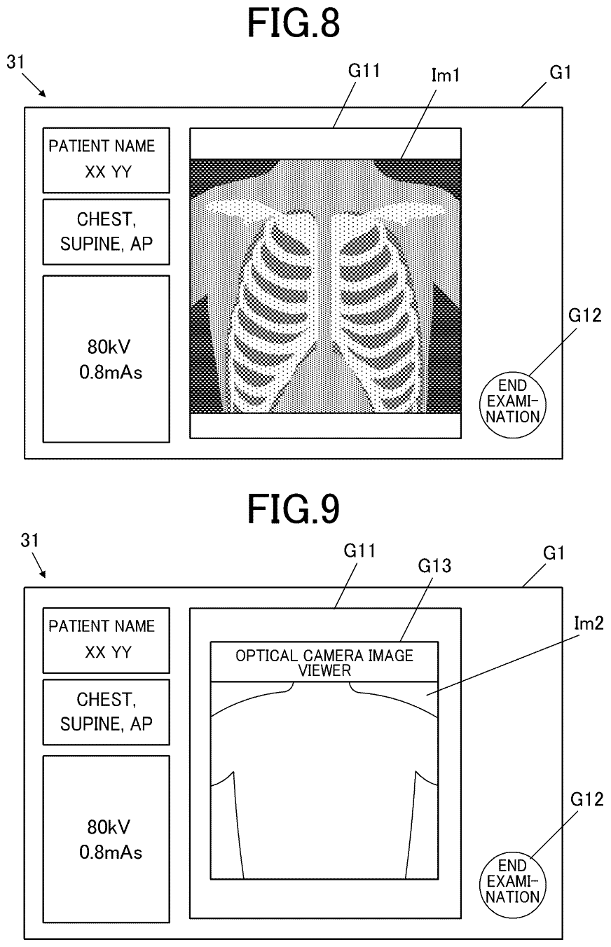

If the controller 211 determines in step S2 that the operation of instructing the start of the examination has been performed (step S2; YES), the controller 211 turns on the display of an optical image imaged by the optical imaging part 2A (step S3). To be specific, as illustrated in FIG. 1, the controller 211 transmits an optical image imaged by the optical imaging part 2A to the tablet 4 via the short-range wireless communication section 215 and causes the display 44 of the tablet 4 to display the optical image. In the example of FIG. 1, an optical image of the subject S is displayed on the display 44 of the tablet 4. Thus, the user U can check the positioning of the subject S on the display 44 of the tablet 4 while assisting the subject S in positioning himself/herself. Note that if the controller 211 determines in step S2 that the operation of instructing the start of the examination has been performed, an examination screen (see FIG. 8) is to be displayed on the main display 31.

Next, the controller 211 determines whether a pressing operation of the irradiation instruction switch 22 has been performed (step S4). If the controller 211 determines in step S4 that the pressing operation of the irradiation instruction switch 22 has not been performed (step S4; NO), the controller 211 repeatedly performs the determination process in step S4.

If the controller 211 determines in step S4 that the pressing operation of the irradiation instruction switch 22 has been performed (step S4; YES), the controller 211 performs radiographic imaging (still image imaging) (step S5). When the radiographic imaging is performed, the FPD 1 accumulates and reads out charges corresponding to radiation that has passed through the subject S as signal values and transmits the read-out signal values to the controller 211 as a radiographic image (an imaged image).

Next, the controller 211 receives the radiographic image transmitted from the FPD 1 via the communication section 214 (step S6). Subsequently, the controller 211 turns off the display of the optical image imaged by the optical imaging part 2A (step S7). To be specific, the controller 211 does not transmit the optical image imaged by the optical imaging part 2A to the tablet 4 and causes the display 44 of the tablet 4 to turn off the display of the optical image.

Next, as illustrated in FIG. 8, the controller 211 displays the radiographic image Im received in step S6 in an image display field G11 of an examination screen G1 displayed on the main display 31 (step S8). Until the radiographic image Im is displayed in the image display field G11, as illustrated in FIG. 9, an optical camera image viewer G13 may be displayed on the examination screen G1, and an optical image Im2 imaged by the optical imaging part 2A may be displayed on the optical camera image viewer G13. In the example of FIG. 9, an optical image of the chest of the subject S is displayed. In this case, for example, in response to a pressing operation of the irradiation instruction switch 22, the optical camera image viewer G13 displayed in the image display field G11 is hidden, and the radiographic image Im is displayed in the image display field G11.

Next, the controller 211 determines whether an operation of instructing the end of the examination, that is, pressing an examination end button G12 (see FIG. 8) provided on the examination screen G1 has been performed (step S9).

If the controller 211 determines in step S9 that the operation of instructing the end of the examination has not been performed (step S9; NO), the controller 211 returns the process to step S3 and repeatedly performs the subsequent processing. On the other hand, if the controller 211 determines in step S9 that the operation of instructing the end of the examination has been performed (step S9; YES), the controller 211 ends the examination process.

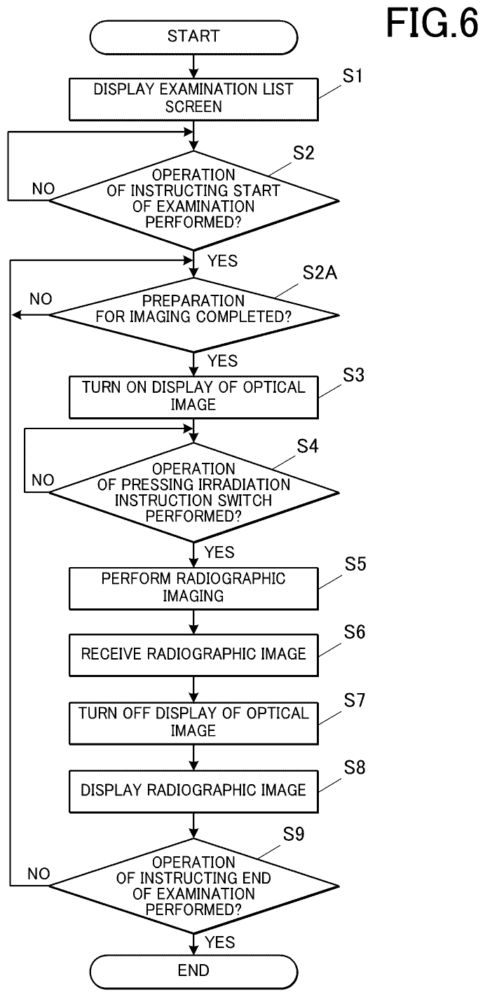

Note that in the examination process illustrated in FIG. 5, if the controller 211 determines that the operation of instructing the start of the examination has been performed (step S2; YES), the controller 211 turns on the display of the optical image imaged by the optical imaging part 2A (step S3). However, this flow is merely an example. For example, as illustrated in FIG. 6, the controller 211 may turn on the display of the optical image imaged by the optical imaging part 2A if the controller 211 determines that the operation of instructing the start of the examination has been performed (step S2; YES) and that a preparation for the radiographic imaging has been completed (step S2A; YES). In this case, an operation flow of the examination process illustrated in FIG. 6 is the same as the operation flow of the examination process illustrated in FIG. 5 except that step S2A for determining whether the preparation for the radiographic imaging has been completed is added between step S2 and step S3.

Therefore, detailed description thereof is omitted.

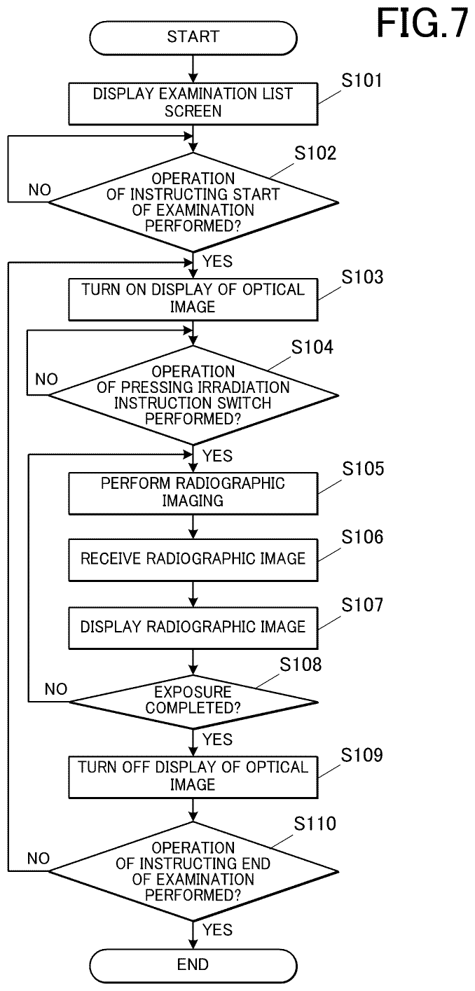

Next, an operation flow of the examination process for moving image imaging (serial imaging) will be described with reference to FIG. 7. Note that since step S101 to step S104 of the operation flow illustrated in FIG. 7 corresponds to step S1 to step S4 of the operation flow illustrated in FIG. 5, description thereof is omitted, and step S105 and subsequent steps will be described. This examination process can also be applied to the case where the system 100 is installed in an imaging room of the medical facility.

If the controller 211 determines in step S104 that the pressing operation of the irradiation instruction switch 22 has been performed (step S104; YES), the controller 211 performs radiographic imaging (moving image imaging) (step S105). When the radiographic imaging (moving image imaging) is performed, each time exposure is performed, the FPD 1 accumulates and reads out charges corresponding to radiation that has passed through the subject S as signal values and transmits the read-out signal values to the controller 211 as a radiographic image (frame image).

Next, the controller 211 receives the radiographic image (frame image) transmitted from the FPD 1 via the communication section 214 (step S106). Subsequently, the controller 211 displays the radiographic image (frame image) received in step S106 in the image display field of the examination screen displayed on the main display 31 (step S107).

Next, the controller 211 determines whether exposure for the moving image imaging has been completed (step S108). If the controller 211 determines in step S108 that the exposure for the moving image imaging has not been completed (step S108; NO), the controller 211 returns the process to step S105, and performs the subsequent processing.

If the controller 211 determines in step S108 that the exposure for the moving image imaging has been completed (step S108; YES), the controller 211 turns off the display of the optical image imaged by the optical imaging part 2A (step S109). To be specific, the controller 211 does not transmit the optical image imaged by the optical imaging part 2A to the tablet 4 and causes the display 44 of the tablet 4 to turn off the display of the optical image. That is, when moving image imaging is performed, the optical image imaged by the optical imaging part 2A is displayed on the display 44 of the tablet 4 during the moving image imaging. As a result, the positioning of the subject S can be checked on the display 44 of the tablet 4 during the moving image imaging. Therefore, when the system 100 is installed in an imaging room of the medical facility, even after the user U leaves the imaging room when performing moving image imaging, the positioning of the subject S during the moving image imaging can be checked on the display 44 of the tablet 4.

Next, the controller 211 determines whether an operation of instructing the end of the examination, that is, pressing the examination end button (see FIG. 8) provided on the examination screen has been performed (step S110).

If the controller 211 determines in step S110 that the operation of instructing the end of the examination has not been performed (step S110; NO), the controller 211 returns the process to step S103 and repeats the subsequent processing. On the other hand, when the controller 211 determines in step S110 that the operation of instructing the end of the examination has been performed (step S110; YES), the controller 211 ends the examination process.

### <3. Effects>

As described above, the system 100 according to the present embodiment includes the optical imaging part (optical camera) 2A that captures an optical image of the subject (subject) S, the radiation generator (control device, hardware processor) 2 (console 3) that displays an imaged radiographic image, and the tablet (mobile terminal) 4 that is wirelessly communicable with the radiation generator 2. The tablet 4 constituting the system 100 includes the display 44 that displays the optical image of the subject S captured by the optical imaging part 2A.

Therefore, according to the system 100, since the optical image of the subject S captured by the optical imaging part 2A can be displayed on the display 44 of the tablet 4, radiographic imaging can be performed while checking the positioning of the subject S with the tablet 4. As a result, the radiographic imaging can be performed immediately after the subject S is positioned. This suppresses a case in which the subject S moves before the radiographic imaging is started after the subject S has been positioned, resulting in re-imaging. Furthermore, since the optical image of the subject S is displayed on the display 44 of the tablet 4 separately from the main display 31, the optical image of the subject S can be displayed without any restrictions. As a result, it becomes easier to check the positioning of the subject S.

### <4. Others>

The above-described embodiment is not intended to limit the present invention and can be modified without departing from the spirit and scope of the present invention.

For example, although the tablet 4 has been described as an example of the mobile terminal constituting the radiographic imaging system according to the present disclosure in the above embodiment, the tablet 4 is merely an example of the mobile terminal. The mobile terminal may be, for example, a personal digital assistant (PDA), a smartphone, a portable monitor that can be carried by the user U, or the like.

In the above embodiment, the tablet 4 is wirelessly connected to the radiation generator 2 (console 3) via the short-range wireless communication section 43. However, the tablet 4 may be wirelessly communicable with the optical imaging part 2A directly.

In the above embodiment, in the operation flows of the respective examination processes in FIGS. 5 and 7, the display of the optical image imaged by the optical imaging part 2A is turned on in response to an operation of instructing the start of the examination. However, the trigger for turning on the display of the optical image imaged by the optical imaging part 2A is not limited to the case where the operation of instructing the start of the examination is performed. For example, the display of the optical image imaged by the optical imaging part 2A may be turned on at a time when a pressing operation of the irradiation instruction switch 22 is performed and the radiographic imaging is started. For another example, the display of the optical image imaged by the optical imaging part 2A may be turned on at a time when a pressing operation of a predetermined button (not illustrated) that is included in the operation part 32 of the console 3 is performed. Furthermore, when the system 100 is installed in an imaging room of the medical facility, the display of the optical image imaged by the optical imaging part 2A may be turned on at a time when the subject S enters the imaging room.

In the above embodiment, when moving image imaging is performed, in a time period during which the display of the optical image imaged by the optical imaging part 2A is turned on, a predetermined viewer may be displayed on the main display 31, and the optical image imaged by the optical imaging part 2A may be displayed on the viewer. Here, the time period during which the display of the optical image imaged by the optical imaging part 2A is turned on is from step S103 to step S109 of the operation flow illustrated in FIG. 7.

In the above embodiment, a case has been described in which the optical camera image viewer G13 is displayed on the examination screen G1 displayed on the main display 31 and the optical image Im2 imaged by the optical imaging part 2A is displayed on the optical camera image viewer G13 (see FIG. 9). Here, the optical image Im2 imaged by the optical imaging part 2A may be displayed, for example, in the image display field G11 of the examination screen G1. In this case, the controller 211 turns off the display of the optical image Im2 at a time when a notification of transfer (transmission) of the radiographic image is received from the FPD 1, a time when a preview image related to the radiographic image is received, or a time when the radiographic image is received. As a result, the positioning of the subject S can be checked until immediately before the radiographic imaging is completed. Note that when the optical image Im2 is displayed on the display 44 of the tablet 4 or the optical camera image viewer G13, the controller 211 may turn off the display of the optical image Im2 at a time when the radiographic imaging is completed or at a time when the examination is completed, in addition to the times described above. Furthermore, when the system 100 is installed in an imaging room of the medical facility, the display of the optical image Im2 may be turned off at a time when the subject S exits the imaging room.

In the above embodiment, when the transition of a focus display of an icon corresponding to the imaging order information and the transition of a focus display of an thumbnail image for checking the imaged radiographic image can be independently performed on the examination screen G1, the imaging order information and the thumbnail image do not correspond to each other after transitioning the focus displays. Therefore, in this case, it is preferable that the optical image Im2 displayed in the image display field G11 is turned off.

In the above embodiment, when the optical image imaged by the optical imaging part 2A is displayed on the display 44 of the tablet 4, information indicating whether the subject S is suitably positioned may be displayed. As an example of the information indicating whether the positioning is suitable or not, an angle (e.g., a roll angle and a pitch angle) of the FPD 1 with respect to the horizontal plane is displayed. In this case, when the suitable positioning is achieved, the color of the numerical value of the angle displayed may be changed. As another example of the information indicating whether the positioning is suitable or not, a simulated image of the FPD 1 in which a display mode (angle) changes according to the angle may be displayed. In this case, when the suitable positioning is achieved, the color of a display frame of the simulated image displayed may be changed. In either case of displaying the angle or the simulated image, correction information on the angle of the FPD 1 with respect to the horizontal plane may be displayed. Instead of the above correction information, correction information for moving the subject S himself/herself to the suitable positioning may be displayed. When the correction information is displayed, correction information on the angle of the tube 23 with respect to the horizontal plane may be displayed. The information indicating whether the subject S is suitably positioned may be output via the sound output section 33. Furthermore, when the region (e.g., the chest) of the subject S imaged by the optical imaging part 2A is different from the imaging region of the imaging order or when the imaging direction of the optical imaging part 2A (e.g., toward the front or the side) is different from the imaging direction of the imaging order, a warning may be issued by the tablet 4. This serves as information indicating consistency between the region of the subject S imaged by the optical imaging part 2A and the imaging region of the imaging order, and consistency between the imaging direction of the optical imaging part 2A and the imaging direction of the imaging order. In addition, information indicating consistency between the subject S imaged by the optical imaging part 2A and the patient information (e.g., gender) related to the subject S may be displayed.

Although embodiments of the present invention have been described and shown in detail, the disclosed embodiments are made for purposes of illustration and example only and not limitation. The scope of the present invention should be interpreted by terms of the appended claims.

What is claimed is:

1. A radiographic imaging system comprising:
an optical camera that captures an optical image of a subject;
a hardware processor that displays an imaged radiographic image; and
a mobile terminal that is wirelessly communicable with the optical camera or the hardware processor, wherein
the mobile terminal includes a display that displays the optical image of the subject captured by the optical camera.

2. The radiographic imaging system according to claim 1, further comprising a medical cart, wherein the radiographic imaging system is movable by the medical cart.

3. The radiographic imaging system according to claim 1, wherein the radiographic imaging system is capable of performing moving image imaging that includes dynamic imaging and fluoroscopic imaging.

4. The radiographic imaging system according to claim 3, wherein the mobile terminal displays, on the display, the optical image of the subject captured by the optical camera during the moving image imaging.

5. The radiographic imaging system according to claim 4, wherein the hardware processor displays the imaged radiographic image during the moving image imaging.

6. The radiographic imaging system according to claim 1, further comprising a radiation generator that emits radiation, wherein the optical camera is provided to a tube of the radiation generator.

7. The radiographic imaging system according to claim 1, wherein the mobile terminal displays the optical image of the subject captured by the optical camera on the display in response to an examination being started.

8. The radiographic imaging system according to claim 1, wherein
the hardware processor determines whether a preparation for imaging of the radiographic image has been completed, and
when the hardware processor determines that the preparation has been completed, the mobile terminal displays the optical image of the subject captured by the optical camera on the display.

9. The radiographic imaging system according to claim 7, wherein the mobile terminal turns off a display of the optical image of the subject on the display in response to imaging of the radiographic image being completed.

10. The radiographic imaging system according to claim 1, wherein the mobile terminal displays, on the display, support information that supports imaging of the radiographic image.

11. The radiographic imaging system according to claim 10, wherein the mobile terminal displays, as the support information on the display,
information indicating consistency with an imaging order and/or patient information based on the optical image of the subject captured by the optical camera, or
information indicating whether the subject is suitably positioned based on the optical image of the subject.

12. A radiographic imaging support method performed by a radiographic imaging system including:
an optical camera that captures an optical image of a subject;
a hardware processor that displays an imaged radiographic image; and
a mobile terminal that is wirelessly communicable with the optical camera or the hardware processor, the method comprising:
displaying the optical image of the subject captured by the optical camera on a display of the mobile terminal.
